(19) United States
(12) Patent Application Publication
(10) Pub. No.: US 2021/0324345 A1
(43) Pub. Date: Oct. 21, 2021

(54) Recombinant Microorganism for Producing 2,3-Butanediol and a Method of Production of 2,3-Butanediol

(71) Applicant: CPC Corporation, Taiwan, Kaohsiung (TW)

(72) Inventors: Hsin-Yao Cheng, Kaohsiung (TW); Chin-Chung Chen, Kaohsiung (TW); Ai-Ling Kao, Kaohsiung (TW); Chang-Ting Tsai, Kaohsiung (TW); Zheng-Chia Tsai, Kaohsiung (TW); Jui-Hui Chen, Kaohsiung (TW); Hwan-Yu Chang, Hsinchu (TW); Li-Ching Kok, Hsinchu (TW)

(21) Appl. No.: 16/936,530

(22) Filed: Jul. 23, 2020

(30) Foreign Application Priority Data
Apr. 21, 2020 (TW) 109113399

(51) Int. Cl. C12N 9/04; C12N 9/12; C12N 9/10; C12N 9/16; C12P 7/18

(52) U.S. Cl. CPC C12N 9/0006; C12Y 207/07009; C12N 9/1241; C12Y 101/01001; C12N 9/1029; C12P 7/18; C12Y 207/08031; C12Y 101/01027; C12N 9/16; C12Y 301/04001; C12N 9/1288

(57) ABSTRACT

A recombinant microorganism for producing 2,3-butanediol consisting of selecting at least three groups from uridine diphosphate glucose phosphate uroglycan transferase gene (galU), acetyl alcohol dehydrogenase gene (acoA), acetyl phosphate transferase gene (pta), adenosine glucosylphosphate transferase gene (glgC), lactose dehydrogenase gene (ldhA), and phosphodiesterase gene (pdeC) which were modified.

### BACKGROUND OF THE INVENTION

### 1. Field of the Invention

The present invention relates to non-naturally occurring microbial organisms capable of producing 2,3-butanediol, wherein the microbial organism includes one or more genetic modifications, and the disclosure further provides methods of producing 2,3-butanediol by using the microbial organisms.

### 2. Description of the Prior Art

2,3-Butanediol is a kind of polyol. It is an important chemical raw material and liquid fuel. Its application range is quite wide, including in different fields such as chemical industry, energy, food and aerospace, with many different uses. For example, in the fuel industry, 2,3-butanediol can be mixed with gasoline as an octane booster or as a liquid fuel. In the chemical industry, 2,3-butanediol has a very low freezing point and can be used as an antifreeze agent. As a multi-purpose chemical raw material, 2,3-butanediol can be converted into 1,3-butadiene through a simple reaction, which can be used as a raw material for synthetic rubber and synthetic resin, or can be converted into methyl ethyl ketone. Except it can be used as a fuel additive and solvent, and also as a low-boiling solvent. It can be widely used in different industries such as adhesives, coatings, fuels, lubricants and inks. In the food industry, 2,3-butanediol can also be converted to 2,3-butanedione (diacetyl) or acetyl alcohol (acetoin), used as a flavoring agent or natural food flavor, both widely used in the food industry.

The methods currently used to produce 2,3-butanediol mainly include chemical methods and biological fermentation methods. The chemical method uses traditional petrochemical methods for cracking and refining, and uses non-renewable fossil crude oil as raw materials. Due to the high temperature and high pressure required in the cracking and refining process, it leads to serious pollution. However, the bio-fermentation method is based on renewable biomass raw materials and is produced by microbial fermentation. Its raw materials and production methods are environmentally friendly, which is more in line with the international community's environmental protection and sustainable development of industry today.

It is known that 2,3-butanediol can be produced by a variety of bacteria, including Klebsiella, Enterobacter, Bacillus, and Serratia, etc. However, after various optimizations for fermentation conditions, such as temperature, pH value, oxygen concentration, etc., and the improvement of microbial fermentation capacity, the production of 2,3-butanediol by microbial fermentation still has a problem of low productivity. Therefore, the cost of producing 2,3-butanediol by the biological fermentation method remains high.

In addition, the above-mentioned microorganisms used for the production of 2,3-butanediol by fermentation are mostly pathogenic, and are classified according to the pathogenic ability of various microorganisms in various health management agencies in various regions. The operation process of using these microorganisms must comply with the corresponding regulations. These regulations and the potential risk of disease during the operation further increase the cost of producing 2,3-butanediol by biological fermentation.

Thus, there is still a need for a safe and high-yield method for the production of 2,3-butanediol, to further reduces the cost of producing 2,3-butanediol by the biological fermentation method.

The present invention has arisen to mitigate and/or obviate the afore-described disadvantages.

### SUMMARY OF THE INVENTION

The primary objective of the present invention is to provide a recombinant microorganism for producing 2,3-butanediol which consisting of selecting at least three groups from uridine diphosphate glucose phosphate uroglycan transferase gene (galU), acetyl alcohol dehydrogenase gene (acoA), acetyl phosphate transferase gene (pta), adenosine glucosylphosphate transferase gene (glgC), lactose dehydrogenase gene (ldhA), and phosphodiesterase gene (pdeC) which had gene modification.

Preferably, the recombinant microorganism further consists of the galU, the acoA, and the pta which had gene modification.

Preferably, the recombinant microorganism further consists of the galU, the acoA, the pta, and the glgC which were modified.

Preferably, the recombinant microorganism further consists of the galU, the acoA, the pta, the glgC, and the IdhA which had gene modification.

Preferably, the recombinant microorganism further consists of the galU, the acoA, the pta, the glgC, the IdhA, and the pdeC which had gene modification.

Preferably, the genetic modification is any one of gene suppression, gene deletion and gene silencing.

Preferably, the genetic modification is gene deletion.

Preferably, the recombinant microorganism is Klebsiella.

Preferably, a yield of 2,3-butanediol of the recombinant microorganism is higher than a yield of 2,3-butanediol of a wild-type microorganism.

Preferably, recombinant microorganism is safer than a wild-type microorganism.

Preferably, a growing rate of the recombinant microorganism is equal to a growing rate of a recombinant microorganism which was not modified.

Preferably, the recombinant microorganism produces the 2,3-butanediol in an acidic environment.

In addition, a method for production of the 2,3-butanediol comprising cultivating the recombinant microorganism and separating the 2, 3-butanediol from the culture medium.

### BRIEF DESCRIPTION OF THE DRAWINGS

FIG. 1A is a schematic view showing a homologous recombination of target gene and DNA fragmentation recombined on chromosomes of wild-type strain and transconjugant according to a preferred embodiment of the present invention.

FIG. 1B is a schematic view showing the target gene on the chromosome after knocking out a mutant strain in two homologous recombinations according to the preferred embodiment of the present invention.

FIG. 2 is a schematic view showing colonies of natural streptomycin-resistant mutants according to the preferred embodiment of the present invention.

FIG. 3 is a schematic view showing the electrophoresis of the deleted gene in S1U1 by ways of PCR amplification according to the preferred embodiment of the present invention.

FIG. 4 is a schematic view showing the electrophoresis of the deleted gene in S1U1D1 by ways of PCR amplification according to the preferred embodiment of the present invention.

FIG. 5 is a schematic view showing the electrophoresis of the gene in S1UID2 by ways of PCR amplification according to the preferred embodiment of the present invention.

FIG. 6 is a schematic view showing the electrophoresis of the deleted gene in S1UID3 by ways of PCR amplification according to the preferred embodiment of the present invention.

FIG. 7 is a schematic view showing the electrophoresis of the deleted gene in S1UID4 by ways of PCR amplification according to the preferred embodiment of the present invention.

FIG. 8 is a schematic view showing the electrophoresis of the deleted gene in S1UID5 by ways of PCR amplification according to the preferred embodiment of the present invention.

FIG. 9 is a schematic view showing colonies of the recombinant gene strains according to the preferred embodiment of the present invention.

FIG. 10 is a diagram showing growth curve lines of the recombinant strains S1U1, S1U1D1, S1U1D2, S1U1D3, and S1U1D4 according to the preferred embodiment of the present invention.

FIG. 11 is a diagram showing growth curve lines of the natural mutant S1 and the recombinant strain S1U1 according to the preferred embodiment of the present invention.

FIG. 12 is a schematic view showing colors of 2,3-BDO with different concentrations tested by TLC-vanillin according to the preferred embodiment of the present invention.

FIG. 13A is a diagram showing a curve line of a gray value tested by TLC-vanillin according to the preferred embodiment of the present invention.

FIG. 13B is a diagram showing a standard curve line of yields of 2,3-BDO of recombinant strains of each gene according to the preferred embodiment of the present invention.

FIG. 14A is a schematic view showing yields of 2,3-BDO of the wild-type strain and the recombinant strain S1U1 cultivated in a M9 medium solution consisting 5% glucose in different times according to the preferred embodiment of the present invention.

FIG. 14B is a schematic view showing yields of 2,3-BDO of the recombinant strain S1U1D1, S1U1D2, S1U1D3, S1U1D4, and S1U1D5 cultivated in the M9 medium solution consisting 5% glucose in different times according to the preferred embodiment of the present invention.

FIG. 15 is a diagram showing a curve line of yields (g/L) of 2,3-BDO of the recombinant strain S1U1, S1U1D1, S1U1D2, S1U1D3, S1U1D4, and S1U1D5 cultivated in the M9 medium solution consisting 5% glucose in different times according to the preferred embodiment of the present invention.

FIG. 16 is a diagram showing a curve line of pH values of leaven of the recombinant strain S1U1, S1U1D1, S1U1D2, S1U1D3, S1U1D4, and S1U1D5 cultivated in the M9 medium solution consisting 5% glucose in different times according to the preferred embodiment of the present invention.

### DETAILED DESCRIPTION OF THE PREFERRED EMBODIMENTS

A genetic modification to a recombinant microorganism according to a preferred embodiment of the present invention means operating genome or nucleic acid of the microorganism, wherein the genetic modification is any one of heterologous gene expression, gene insertion or promoter insertion, gene deletion or gene silencing, a change of gene expression or inactivation of gene expression, gene suppression, enzyme engineering, directed evolution, knowledge-based design, induction of random mutation, gene shuffling, and codon optimization, etc.

With reference to Sequence Listings 1 and 5, sequences disclosed are primer sequences.

A recombinant microorganism for producing of 2,3-butanediol according to the preferred embodiment consists of selecting at least three groups from uridine diphosphate glucose phosphate uroglycan transferase gene (galU), acetyl alcohol dehydrogenase gene (acoA), acetyl phosphate transferase gene (pta), adenosine glucosylphosphate transferase gene (glgC), lactose dehydrogenase gene (ldhA), and phosphodiesterase gene (pdeC) which had the genetic modification.

An experimental method of the present invention comprises steps of:

1) cultivating strains, wherein the strains are cultivated at a Luria-Bertani (LB) or Yeast Extract Peptone (YPD) culture medium for 18 hours to 24 hours in a room temperature or in a temperature of 30° C.;

2) extracting whole-cell chromosome, wherein the whole-cell chromosome is drawn so as to be used as a template for amplifying a DNA fragmentation consisting of a target gene, and a whole-cell chromosomal DNA is provided to be preserved in a long period of time in the step of extracting the whole-cell chromosome by ways of a DNA isolation kit, such as Qiagen DNeasy Plant Mini Kit;

wherein when extracting the whole-cell chromosome, 2 ml of bacteria is centrifugally collected and cultivates in a LB culture medium for 40 hrs, and each sample is re-dissolved in 400 μL AP1 buffer solution preheated to 65° C., wherein the 400 μL AP1 buffer solution consists of 4 uL RNaseA (Genomic DNA Kit for Plant Tissues, Yeastern Biotech Co., Ltd.) and is shocked, suspends, and stirred in a water bath in a temperature of 65° C. for 5 minutes, thereafter 130 uL buffer solution is added, stirred, and cooled in the water bath for 5 minutes, then the 130 uL buffer solution is centrifuged at a rotation speed of 14,000 rpm for five minutes, supernatant is drawn to a QIAshredder spin column and is accommodated in a 2 ml collection tube and is centrifuged at a rotation speed of 14,000 rpm for two minutes, and filtrate is moved to a new spin column, wherein when precipitated substance occurs in the spin column, the precipitated substance is not stirred and AW1 buffer solution is added to the spin column and is mixed evenly with the precipitated substance after being drawn by a microcentrifuge column, then 650 uL of mixed liquid is extracted to a DNeasy Mini spin column and is accommodated in the 2 ml collection tube and is centrifuged at a rotation speed of 8,000 rpm for one minute. Thereafter, the filtrate is poured until the mixed liquid is filtered. The 2 ml collection tube is replaced by a lower collection tube, and 500 uL of AW2 buffer solution is added to an upper spin column and is centrifuged at a rotation speed of 8,000 rpm for one minute, wherein filtrate is poured out of the upper spin column and the AW2 buffer solution washes the upper spin column repeatedly, then the AW2 buffer solution is centrifuged at a rotation speed of 14,000 rpm for two minutes, the filtrate is poured out of the upper spin column, and the upper spin column is removed, wherein the filtrate remains on an inner wall of the upper spin column, and a 1.5 ml microcentrifuge column is replaced and adds and puts 100 uL AE buffer solution (i.e., 10 mM Tris-HCl, 0.5 mM EDTA in pH 9.0) in the 1.5 ml microcentrifuge column for five minutes in the room temperature (wherein the 100 uL AE buffer solution is preheated in a temperature of 65° C. before being added), thereafter the 1.5 ml microcentrifuge column is centrifuged at a rotation speed of 14,000 rpm for one minute, thus finishing extraction of the whole-cell chromosome. The whole-cell chromosome is analyzed and is measured by a spectrophotometer, wherein a blank control group is the AE buffer solution, and each sample is extracted in 4 uL and is diluted by adding 400 uL AE buffer solution, then 260 nm and 280 nm absorbances are measured respectively.

The experimental method of the present invention comprises steps of:

3) having polymerase chain reaction (PCR), wherein polymerase in the PCR is Phusion High Fidelity DNA Polymerase (Thermo Scientific, Vilnius, Lithuania, USA), and the PCR includes sub-steps of:

reacting in a temperature of 95° C. for five minutes, and repeating 35-40 times the following sub-steps: reacting for 30 seconds to 3 minutes in a temperature of 90° C. (when a colony is the template, the 30 seconds to 3 minutes are prolonged to ten minutes), reacting 30 seconds in a temperature of 50° C. to 62° C. (which is set by a gradient or is changed based on experimental requirements), reacting 90 seconds to 120 seconds in a temperature of 72° C. (or is set to 15 secs/kb to 30 secs/kb based on a length of an amplified fragmentation). Thereafter, the polymerase chain reaction (PCR) is executed for 5 minutes to 10 minutes in a temperature of 72° C. and is cooled to a temperature of 4° C.

4) analyzing electrophoresis of mixture in the PCR, wherein 10 uL PCR mixture is electrophoresis analyzed by ways of 1% agarose gel in 135 voltages, the 10 uL PCR mixture is dyed for 30 minutes by 0.5 uL/mL ethidium bromide, and the 10 uL PCR mixture is decolorized by primary water (distilled water) for ten minutes and is analyzed and taken pictures by UV light imaging.

5) having homologous recombination to knock out gene, wherein an upstream sequence and a downstream sequence of the target gene are recombined homologously and are counter-selected, wherein after having a second homologous recombination, recombinant strain of plasmid sequence consisting of antibiotic resistance gene loses, wherein the selected strain does not have drug resistance, because the antibiotic resistance gene of the selected strain disappears with plasmid loss. When a sequence of a wild-type strain remains on a chromosome after two homologous recombinations, the strain recovers to the wild-type strain after plasmid loss which is called as revertant.

Thereby, it is stable to have homologous recombination so as to knock out the gene, and it is possible to select a mutant. To have a counter-selection, drug-resistant strains of streptomycin are selected randomly to obtain mutate strain, thus changing physiological characteristics of a part of strains after being selected randomly. Furthermore, the upstream sequence and the downstream sequence of the target gene consist of 1000 bp fragmentation configured to construct the mutant plastid, thus causing mutation. A mutant target strain receives a gene delivery by ways of a conjugation.

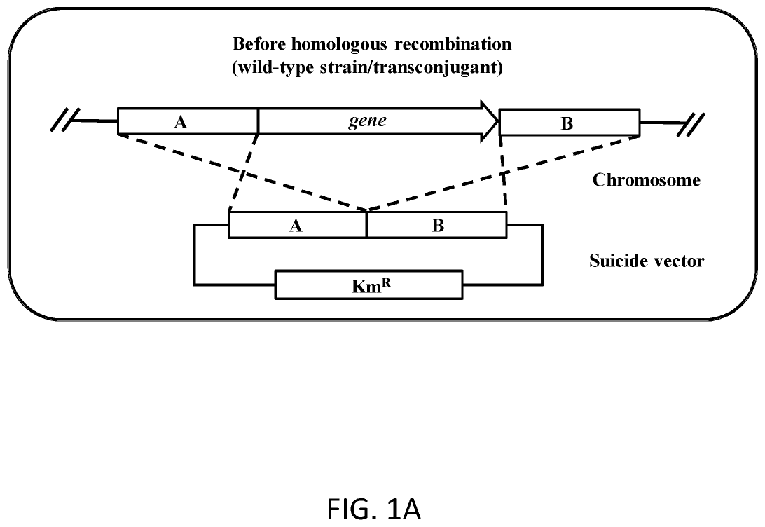

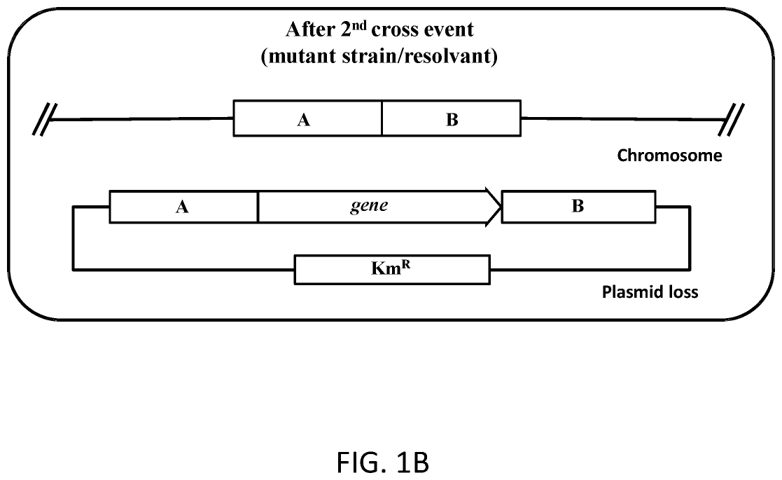

Taking FIG. 1A for example, before homologous recombination, the target gene on chromosome of the wild-type strain and the transconjugant are homologously recombined with the DNA fragmentation and are counter-selected, wherein the mutation are represented by A and B on an upstream area and a downstream area of a suicide plastid. Referring to FIG. 1B, after the two homologous recombinations, the target gene on the chromosome of the mutant strain is plasmid loss, wherein KMR denotes the drug resistance gene of kanamycin.

A method of constructing recombinant strain by using gene loss of homologous recombination comprises steps of: (a) selecting a natural mutant strain from the streptomycin, (b) selecting a first homologous recombination strain by using the conjugation, (c) counter-selecting to acquire a second homologous recombination strain, and (d) confirming al gene mutation location.

When selecting the natural mutant from the streptomycin, a single colony of the wild-type strain is inoculated into 2 mL LB culture solution, and the 2 mL LB culture solution is rotated for 8 hours in a temperature of 37° C., then the 2 mL LB culture solution is centrifuged to collect bacteria. Thereafter, the bacteria are washed two times with physiological saline (0.85% NaCl), coated on the LB culture medium consisting of 500 μg/mL streptomycin, and cultivated overnight in the temperature of 37° C.

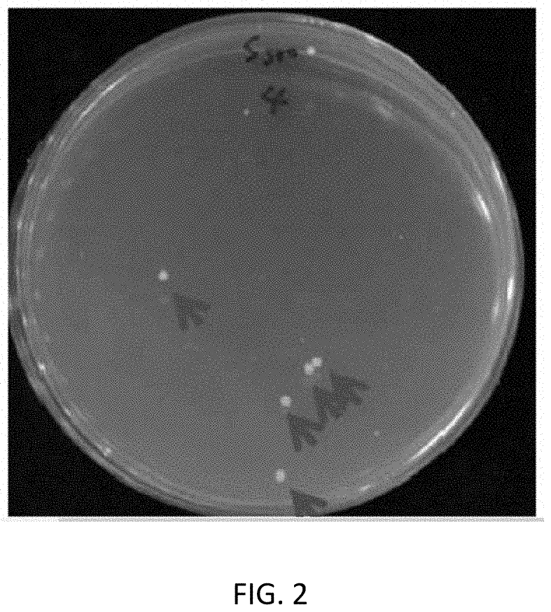

FIG. 2 is a schematic view showing colonies of natural mutants of streptomycin, wherein the colonies of LB culture medium capable of growing with 500 μg/mL streptomyces are selected (shown by arrows of FIG. 2), and a single colony is selected and is cultivated in LB medium consisting of 500 jug/mL streptomycin and is preserved in a temperature of −80° C., wherein the streptomycin is a natural mutant S1.

Thereafter, the natural mutant S1 of the streptomycin is conjugated, and the first homologous recombination strain is selected. In the conjugation, the mutant plastid is sent into the streptomycin natural mutant, for example, a donor and a recipient are mixed at a predetermined proportion and are cultivated, and colonies with streptomycin resistance are selected, wherein PCR is configured to confirm whether the gene fragmentation is successfully embedded in the target gene location. In the second homologous recombination, a single colony is selected to be cultivated, diluted, and coated on LB culture medium consisting of the streptomycin, and a different single colony is selected, coated on LB culture medium consisting of kanamycin or the streptomycin, and cultivated overnight, then the colonies tolerating streptomycin but losing kanamycin resistance are selected and confirmed by using PCR, such that the colonies are gene-deletion mutant or revertant and are electrophoresis analyzed.

The experimental method of the present invention comprises steps of:

6) measuring 2,3-butanediol (2,3-BDO), wherein compositions of bacterial culture is separated by a thin layer chromatography and is coloured with vanillin, wherein reaction conditions include:

dropping 5 μL sample onto one end of Pre-activated silica TLC plate of the thin layer chromatography (TLC), and having chromatographic analysis by using hexane:ethyl acetate:glacial acetic acid=70:30:1.5 as mobile phase. After 40 minutes, the mobile phase is close to a top of the Pre-activated silica TLC plate, and display agent (vanillin:sulfuric acid:ethanol=0.5 g:1 ml:9 ml; Sigma-Aldrich) is sprayed to the pre-activated silica TLC plate and is baked in a temperature of 110° C. for five minutes to observe the color. After testing, only 2,3-BDO appears blue, and glucose are dark brown. Others (such as acetyl alcohol and diacetyl) do not have color.

In a first embodiment of the present invention, the mutant plastids are constructed.

The upstream sequence and the downstream sequence of the target gene of the amplified polymerase chain are DNA fragmentation of 1,000 base pair (bp), and primer sequence in the polymerase chain reaction is shown in Sequence Listing 1.

Sequence Listing 1 - Primer sequence in
polymerase chain reaction

| Primer | Primer | SEQ ID |
|---|---|---|
| name | sequence | NO. |
| acoA_up | ATGAATTCTGAAGCGATCTTCATGCCC | SEQ ID |
| (F) | | NO. 1 |
| acoA_up | ATTCTAGAAAGGTCACCCAGGCGGG | SEQ ID |
| (R) | | NO. 2 |
| acoA_down | ATTCTAGACGCTGCCTACCCGGCTC | SEQ ID |
| (F) | | NO. 3 |
| acoA_down | ATGATATCAGCACTTGACGGACGGC | SEQ ID |
| (R) | | NO. 4 |
| glgC_up | ATGAATTCACTGTTCGAGGCTATCCGC | SEQ ID |
| (F) | | NO. 5 |
| glgC_up | ATGGTACCCGGATCGTTTTTTTCAAGC | SEQ ID |
| (R) | | NO. 6 |
| glgC_down | ATGGTACCAAACAGGAGCGCTAATGC | SEQ ID |
| (F) | | NO. 7 |
| glgC_down | ATTCTAGAGCCCACTTTGCCTGGATGT | SEQ ID |
| (R) | | NO. 8 |
| ldhA_up | ATGATATCTAAGACGCGGGCTCTCCTG | SEQ ID |
| (F) | | NO. 9 |
| ldhA_up | ATGGTACCCCGCGATTTTCATAAGACT | SEQ ID |
| (R) | | NO. 10 |
| ldhA_down | ATGGTACCCTGATCAGCATTTCGGAGA | SEQ ID |
| (F) | | NO. 11 |
| ldhA_down | ATGAATTCTACTTCCCCTCTCGACGCC | SEQ ID |
| (R) | | NO. 12 |
| pdeC_up | TATCTAGAGGCTGCAGAAAACGAAAAAGC | SEQ ID |
| (F) | | NO. 13 |
| pdeC_up | TAGGATCCACCATTTCCGTTTTTTGC | SEQ ID |
| (R) | | NO. 14 |
| pdeC_down | ATGGATCCCTCTACAAGCGCGGCGTAC | SEQ ID |
| (F) | | NO. 15 |
| pdeC_down | ATGAATTCCTCGCCTGCAGACAAAAC | SEQ ID |
| (R) | | NO. 16 |

The upstream and the downstream fragmentations of the target genes are conjugated and knocked in the suicidal plastid pKAS46 to construct the mutant plastid with different fragmentation of target gene, as shown in Table 2.

TABLE 2
Knocked suicidal plastid after conjugating the upstream
and downstream fragmentations of the target genes.

| Plastid | |
|---|---|
| name | Description |
| pKAS46-D1 | Selected plastid pKAS46 consisting of |
| | upstream 998 bp and downstream 1076 bp |
| | of sequence of gene acoA |
| pKAS46-D3 | Selected plastid pKAS46 consisting of |
| | upstream 1028 bp and downstream 1064 bp |
| | of sequence of gene glgC |
| pKAS46-D4 | Selected plastid pKAS46 consisting of |
| | upstream 1025 bp and downstream 1020 bp |
| | of sequence of gene ldhA |
| pKAS46-D5 | Selected plastid pKAS46 consisting of |
| | upstream 1000 bp and downstream 500 bp |
| | of sequence of gene pdeC |

Furthermore, the mutant plastid of the target gene galU is provided by associate professor Lai Yiqi of Chung Shan Medical University, wherein the mutant plastid consists of the gene galU with knockout out 710 bp and is named as pYC094 because of pKAS46 gene selection plastid with 1.8 kb DNA fragmentation.

A method of constructing mutant plastids with target gene pta is to amplify 1700 bp fragmentation by using the PCR (the primer sequence of PCR is pta (F)): TCTAGACATCTTCCATCTGCACGACACCC (SEQ ID NO. 29) and pta (R) GAATTCAGTCGGCGTTGATGTAGTTGGC (SEQ ID NO. 30)), and a middle of the fragmentation is cut into 300 base pairs by restriction enzyme Kpni, then the suicide plastid pKAS46 (called as pKAS46-D2) is conjugated as listed in Table 3.

TABLE 3
Comparison pG-D2 with pKAS46-D2

| Plastid | |
|---|---|
| name | Description |
| pG-D2 | Plastid pGem-T easy consisting of upstream |
| | 566 bp and downstream 1192 bp of |
| | sequence of gene pta is cut and conjugated |
| | by restriction enzyme KpnI. |
| pKAS46-D2 | Selected Plastid pKAS46 having Plastid |
| | pG-D2 consisting of 1453 bp fragmentation |

In a second embodiment of the present invention, the recombinant strain is constructed.

The mutant plastids of the first emdboimdnet are transformed and send to E. coli S17-1 λpir to produce E. coli strain, and the recombinant strain consisting of the target gene is produced by gene knockout of the homologous recombination.

For instance, the natural mutant S1 of the streptomycin is the recipient strain in the conjugation, and a give strain is E. coli S17-1 λpir consisting of recombinant plastid for pYC094 mutation (kanamycin and ampicillin), the colonies are cultivated overnight in the LB medium consisting of antibiotic and are centrifuged to collect the bacteria. The bacteria are washed two times with physiological saline, mixed at a proportion 2:1 (give strain: recipient strain), centrifuged, and coated on nitrocellulose membrane (NC membrane) of the LB culture medium, wherein the bacteria are cultivated overnight in the temperature of 37° C., and the NC membrane is selected and is put into a test tube with 3 mL LB culture solution by sterile tweezers. After shocking the test tube so that the bacteria remove from NC membrane and move to the LB culture solution, 1 mL re-suspend bacterial solution is drawn and is centrifuged to collect the bacteria. Then, the bacteria are washed two times with the physiological saline and are diluted to 10-fold sequence (10 times and 100 times dilution) in the physiological saline, wherein 100 μL bacterial solution with different dilution ratio are coated on M9 culture medium (consisting of 47 mM Na₂HPO₄, 22 mM KH₂PO₄, 18 mM NH₄Cl, 8 mM NaCl, 2 mM MgSO₄, and 0.15 mM CaCl₂)), LB culture medium (consisting of kanamycin and ampicillin), and M9 culture medium (consisting of kanamycin and ampicillin) and are cultivated overnight in the temperature of 37° C. Thereafter, the colonies capable of growing in the M9 culture medium are selected and are purified in the LB culture medium (consisting of kanamycin and ampicillin). In the meantime, the colonies are transconjugant in the first homologous recombination. Then, the PCR is configured to confirm whether the gene fragmentation is successfully embedded in the target gene location.

In the second homologous recombination, a single colony is selected and is cultivated in the LB culture solution overnight in the temperature of 37° C., and the bacterial solution cultivated overnight is diluted 100 times into the LB culture solution consisting of 500 μg/mL streptomycin, rotatably cultivated for 8 hours, diluted with the sequence of physiological saline, coated on the LB culture solution consisting of 500 μg/mL streptomycin, and is cultivated overnight in the temperature of 37° C. Thereafter, a different single colony is selected, coated on the LB culture medium consisting of kanamycin or streptomycin, and is cultivated overnight so as to select the colony which tolerate streptomycin but lose kanamycin resistance, the colonies are confirmed by PCR, wherein the colonies are the mutant strain or a wild-type revertant after being electrophoresis analyzed, and the mutant strain is recombinant strain with deletion of galU gene.

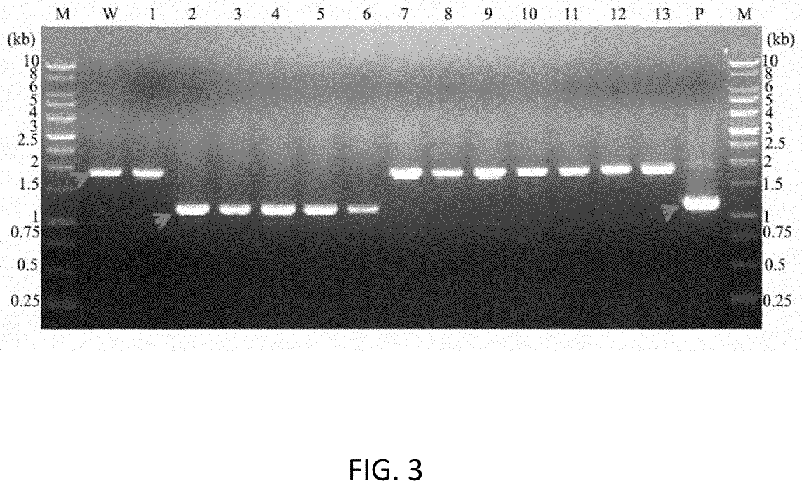

For example, using PCR primer pair p032: GCCGAGCTCACTCTTGCATGGATGGCT (SEQ ID NO. 17) and p042: GTCAGCTGAATTTCATCAC (SEQ ID NO. 18) to execute PCR to galU gene fragmentation, and 1% colloid is produced by ways of buffer solution of 1×TAE (Tris-base, acetic acid, and Ethylenediaminetetraacetic acid (EDTA) and is electrophoresed at 90 voltages for 40 minutes, wherein the target gene fragmentation of the selected strain in the second homologous recombination is analyzed as shown in FIG. 3, five colonies No. 2 to 6 are gene-deletion mutant, named as S1U1, and colonies No. 1 and 7 to 6 are revertant, M is DNA ladder with 1 kb, W represents amplification result of genome DNA of 51 strain cultured overnight, P is amplification result of pYC094 plastid DNA, and fragmentation of the wild-type strain and fragmentation of the mutant strain amplified by the PCR are represented by arrows respectively.

The strains consisting of mutant plastid with different target genes obtained in the first embodiment are used as recipient strains or give strains in conjugation after the gene knockout of the second homologous recombination are executed so as to construct recombinant strains by modifying one or more target genes, as listed in Table 4.

Table 4 shows plastid strains with different target genes and recipient strains in the conjugation, and the recombinant strains after conjugation and selection.

| | | Name of | |
|---|---|---|---|
| | | recombinant | |
| Give | Recipient | strain after | Target gene modification |
| strain | strain | selection | of Recombinant strain |
| E. coli strain | S1U1 | S1U1D1 | galU, acoA |
| transformed with | | | |
| pKAS46-D1 | | | |
| E. coli strain | S1U1D1 | S1U1D2 | galU, acoA, pta |
| transformed with | | | |
| pKAS46-D2 | | | |
| E. coli strain | S1U1D2 | S1U1D3 | galU, acoA, pta, glgC |
| transformed with | | | |
| pKAS46-D3 | | | |
| E. coli strain | S1U1D3 | S1U1D4 | galU, acoA, pta, glgC, |
| transformed with | | | ldhA |
| pKAS46-D4 | | | |
| E. coli strain | S1U1D4 | S1U1D5 | galU, acoA, pta, glgC, |
| transformed with | | | ldhA, pdeC |
| pKAS46-D5 | | | |

After selecting the recombinant strains S1U1D1, S1U1D2, S1U1D3, S1U1D4, and S1U1D5, the target gene fragmentations are amplified by the PCR, and PCR primer are listed in Table 5.

Table 5—shows the sequence of each PCR primer and SEQ ID NO.

| Primer | | |
|---|---|---|
| name | Primer sequence | SEQ ID NO. |
| acoA_check | CGTGGAAGTCGTCGATAATCAGGTAC | SEQ ID NO. |
| (F) | | 19 |
| acoA_check | AACTTAGCCGCCTGGTTGTACAGTGC | SEQ ID NO. |
| (R) | | 20 |
| pta_check | TCTAGACATCTTCCATCTGCACGACACCC | SEQ ID NO. |
| (F) | | 21 |
| pta_check | GAATTCAGTCGGCGTTGATGTAGTTGGC | SEQ ID NO. |
| (R) | | 22 |
| glgC_check | TAATGCTTACTGCCAGGACAATGCCC | SEQ ID NO. |
| (F) | | 23 |
| glgC_check | CCATGCATGTTGTAAAACGACCACG | SEQ ID NO. |
| (R) | | 24 |
| ldhA_check | AGCCTCGGTCATTTCCTGCTAATGTG | SEQ ID NO. |
| (F) | | 25 |
| ldhA_check | AGCGTCAACTGGTTTTCCGTCAGATC | SEQ ID NO. |
| (R) | | 26 |
| pdeC_check | CGTAATCGCTTTTGCGAAGCTGAATA | SEQ ID NO. |
| (F) | | 27 |
| pdeC_check | GGCTACGTCTCGCAGCAAACCTTCT | SEQ ID NO. |
| (R) | | 28 |

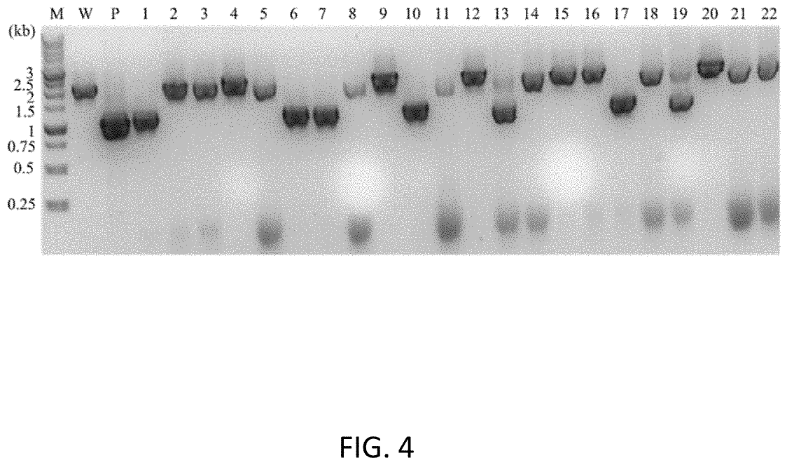

The fragmentations of the PCR are electrophoresis analyzed as shown in FIGS. 4-8.

FIG. 4 is a schematic view showing the electrophoresis of the gene S1U1 by ways of RT-PCR, wherein seven colony nos. 1, 6, 7, 10, 13, 17, and 19 with a fragmentation size 1094 bp are gene-deletion mutant, and fifteen colony nos. 2-5, 8-9, 11-12, 1-16, 18, and 20-22 with a fragmentation size 2048 bp are revertant. M is DNA ladder with 1 kb, W represents amplification result of genome DNA of S1U1 strain cultured overnight, and P is amplification result of pKAS46-D1 plastid DNA.

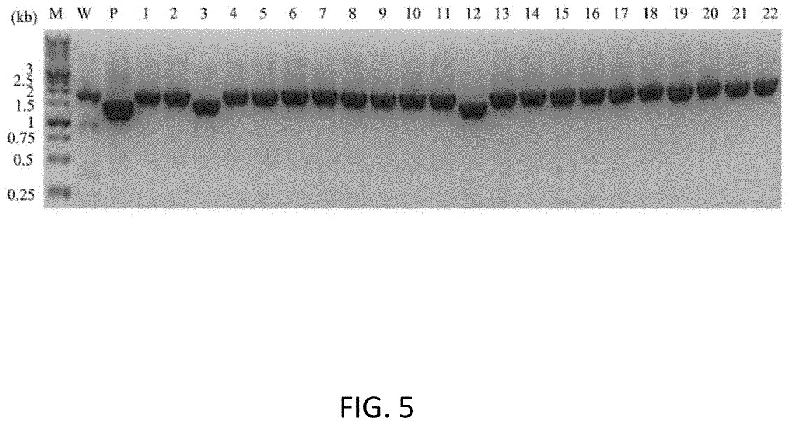

FIG. 5 is a schematic view showing the electrophoresis of the gene S1UID2 by ways of RT-PCR, wherein two colony nos. 3 and 12 with a fragmentation size 1453 bp are gene-deletion mutant, and twenty colony nos. 1-2, 4-11, and 13-22 with a fragmentation size 1758 bp are revertant. M is DNA ladder with 1 kb, W represents amplification result of genome DNA of S1U1 strain cultivated overnight, and P is amplification result of pKAS46-D2 plastid DNA.

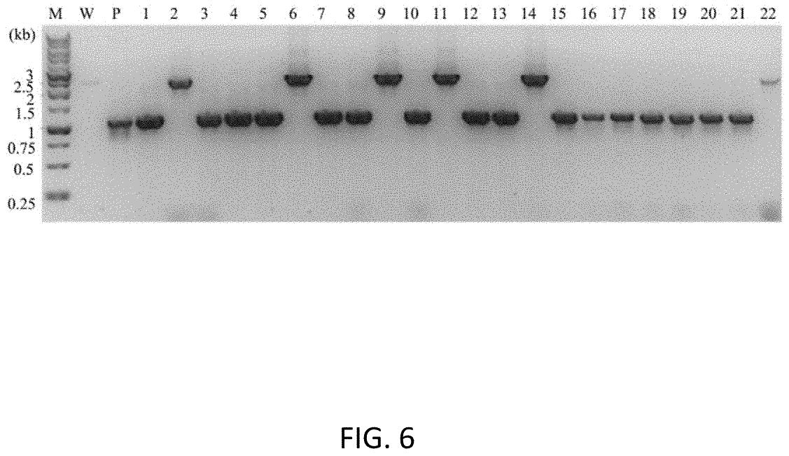

FIG. 6 is a schematic view showing the electrophoresis of the gene S1UID3 by ways of RT-PCR, wherein sixteen colony nos. 1, 3-5, 7-8, 10, 12-13, and 15-21 with a fragmentation size 1083 bp are gene-deletion mutant, and six colony nos. 2, 6, 9, 11, and 14-22 with a fragmentation size 2337 bp are revertant. M is DNA ladder with 1 kb, W represents amplification result of genome DNA of S1U1 strain cultivated overnight, and P is amplification result of pKAS46-D3 plastid DNA.

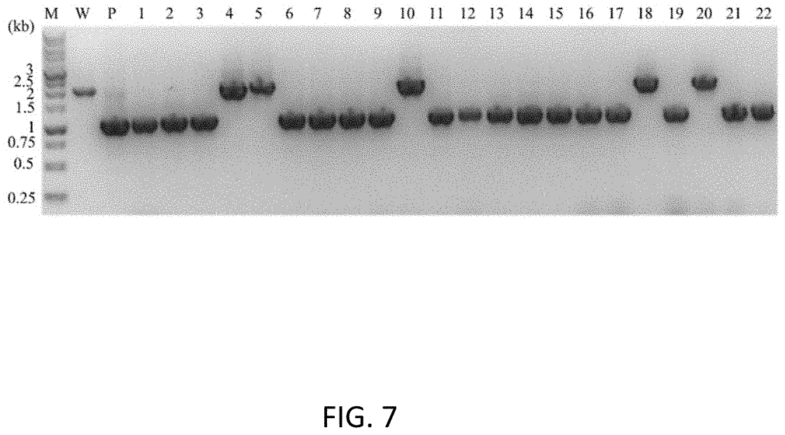

FIG. 7 is a schematic view showing the electrophoresis of the gene S1UID4 by ways of RT-PCR, wherein sixteen colony nos. 1, 3-5, 7-8, 10, 12-13, and 15-21 with a fragmentation size 1083 bp are gene-deletion mutant, and six colony nos. 2, 6, 9, 11, and 14-22 with a fragmentation size 2337 bp are revertant. M is DNA ladder with 1 kb, W represents amplification result of genome DNA of S1U1 strain cultivated overnight, and P is amplification result of pKAS46-D4 plastid DNA.

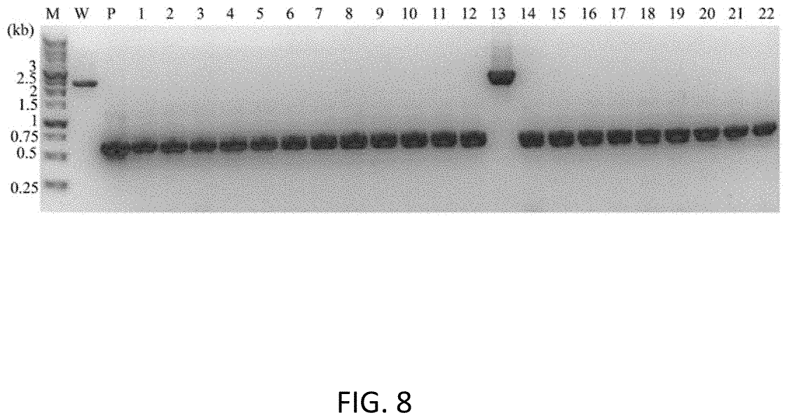

FIG. 8 is a schematic view showing the electrophoresis of the gene S1UID5 by ways of RT-PCR, wherein twenty-one colony nos. 1-12 and 14-22 with a fragmentation size 600 bp are gene-deletion mutant, and a colony no. 13 with a fragmentation size 2551 bp is revertant. M is DNA ladder with 1 kb, W represents amplification result of genome DNA of S1U1 strain cultivated overnight, and P is amplification result of pKAS46-D5 plastid DNA.

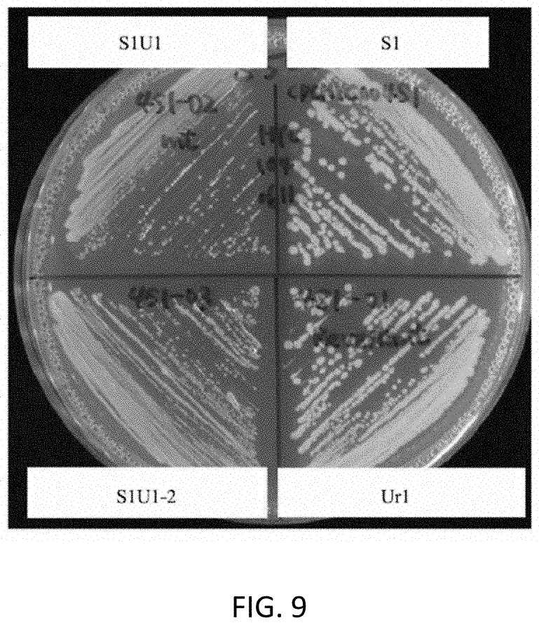

FIG. 9 is a schematic view showing colonies of the recombinant gene strains. The recombinant gene strains S1UI and S1U1-2 are compared with the natural mutant 51 which is not modified, wherein the important gene galU lacking of synthetic capsular polysaccharide has small colonies, and appearance of the revertant Ur 1 is similar to the natural mutant S1.

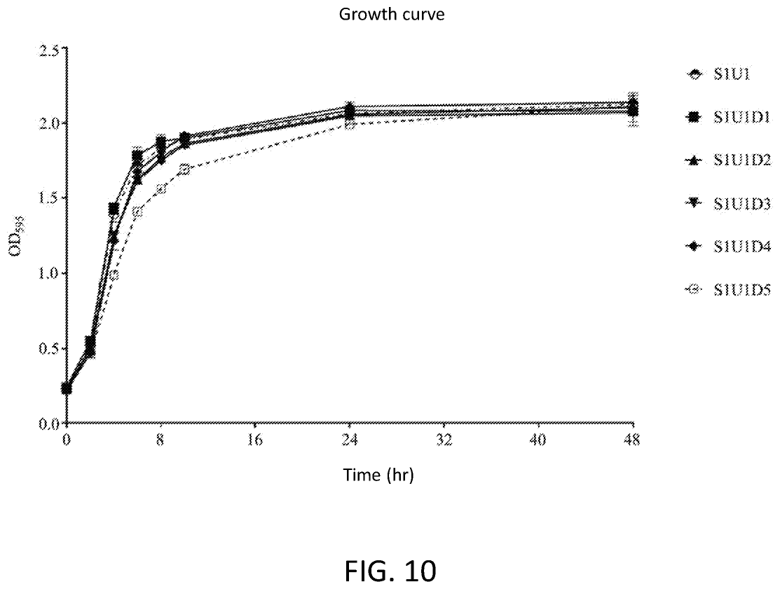

In a third embodiment, growth conditions of the recombinant strain are compared, wherein the natural mutant S1 and the recombinant strains S1U1, S1U1D1, S1U1D2, S1U1D3, S1U1D4, and S1U1D5 are cultivated in M9 culture medium consisting of 5% glucose and is shocked in a rotation speed of 200 rpm in a temperature of 30° C., wherein an absorbance value of a bacterial solution OD595 is measured for every two hours. FIG. 10 is a diagram showing a growth curve of the recombinant strains S1U1, S1U1D1, S1U1D2, S1U1D3, and S1U1D4, wherein growing speeds of the recombinant strains S1U1, S1U1D1, S1U1D2, S1U1D3, and S1U1D4 are similar, but the recombinant strain S1U1D5 grows more slowly than other recombinant strain in first 30 hours, and its growing speed is similar to the growing speeds of the other recombinant strains.

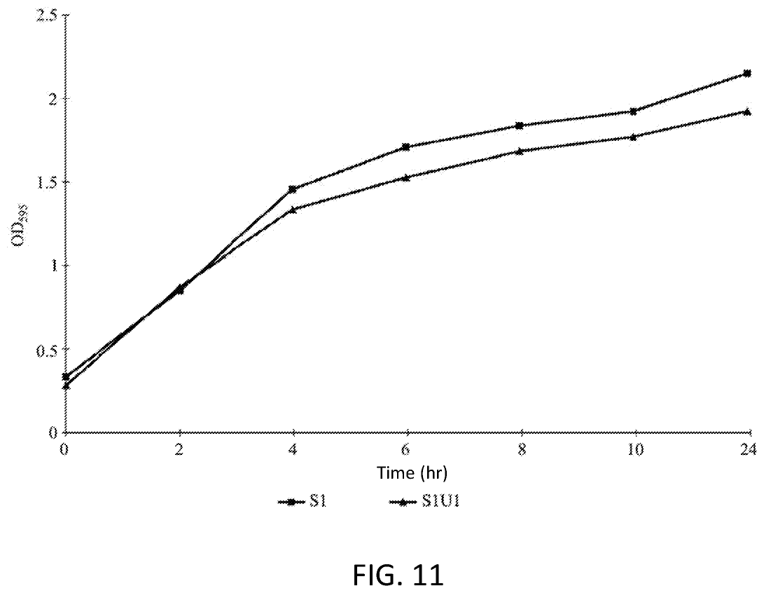

FIG. 11 is a diagram showing growth curves of the natural mutant S1 and the recombinant strain S1U1, wherein the growth curves of the natural mutant S1 and the recombinant strain S1U1 are not different obviously, and after the recombinant strain S1U1 grows for at least four hours, it grows slowly. In other words, the growing speeds of the recombinant strains are not influenced by the modified gene.

In a fourth embodiment, yields of polyols produced by leavens of recombinant strains are compared.

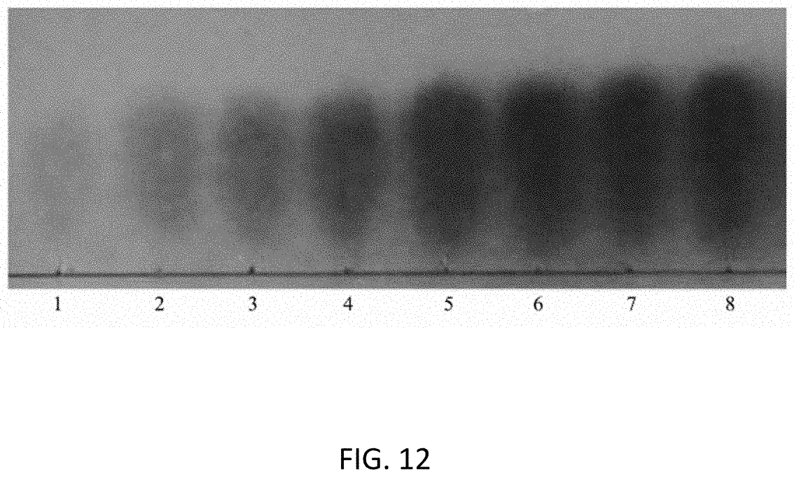

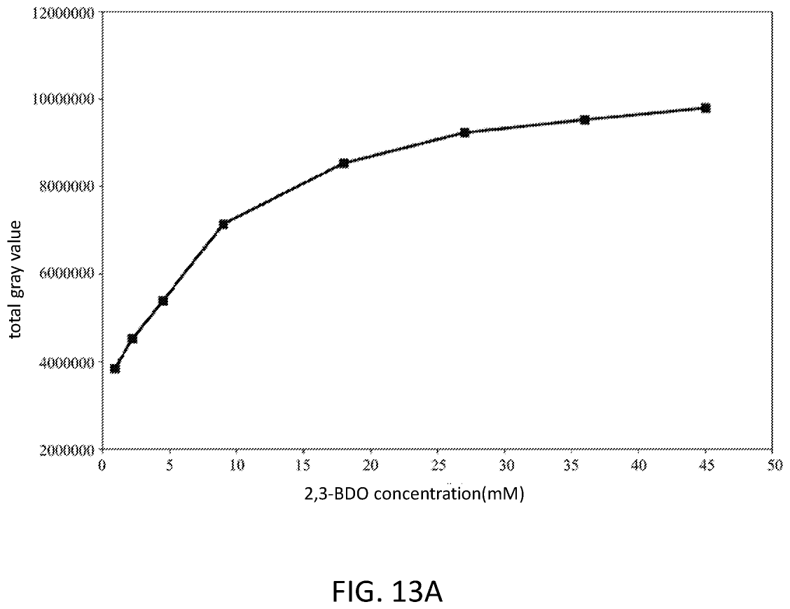

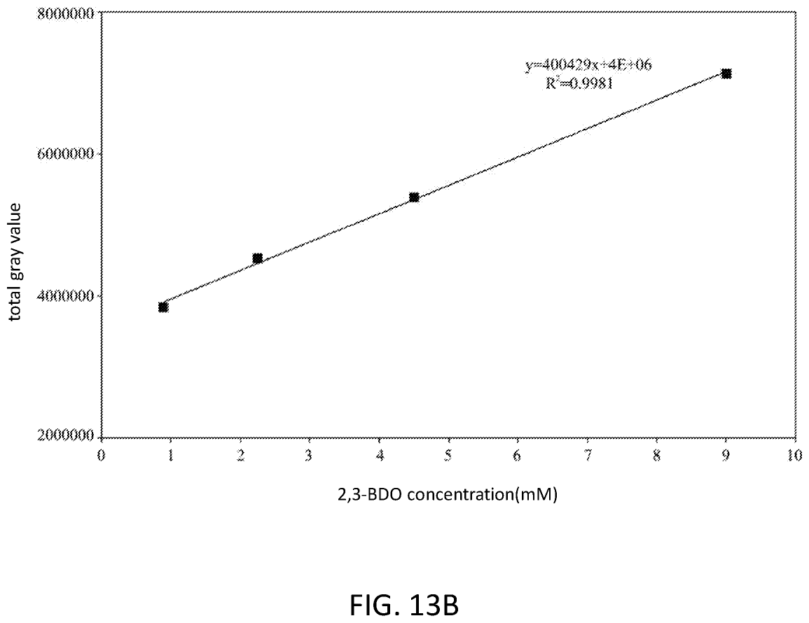

A TLC-vanillin test is executed by different concentrations of 2,3-BDO (such as 10 mM, 25 mM, 50 mM, 100 mM, 200 mM, 300 mM, 400 mM, 500 mM), and an image J software is configured to transform image into gray scale as shown in FIG. 12, and a regression curve is drawn in Excel, as illustrated in FIG. 13, wherein the regression curve is a standard curve line configured to calculate a yield of 2,3-BDO of each mutant strain.

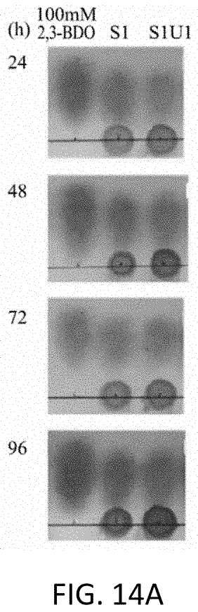

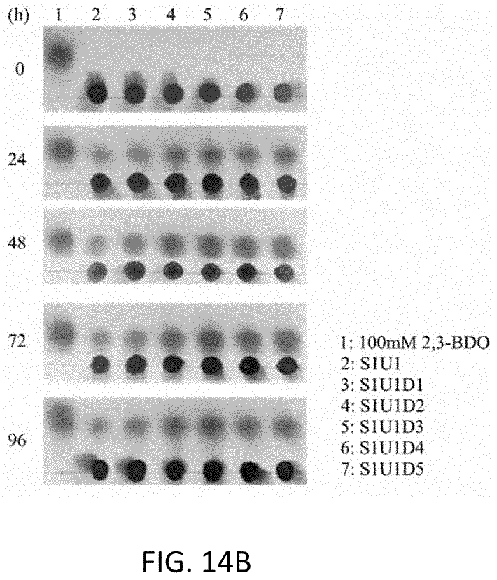

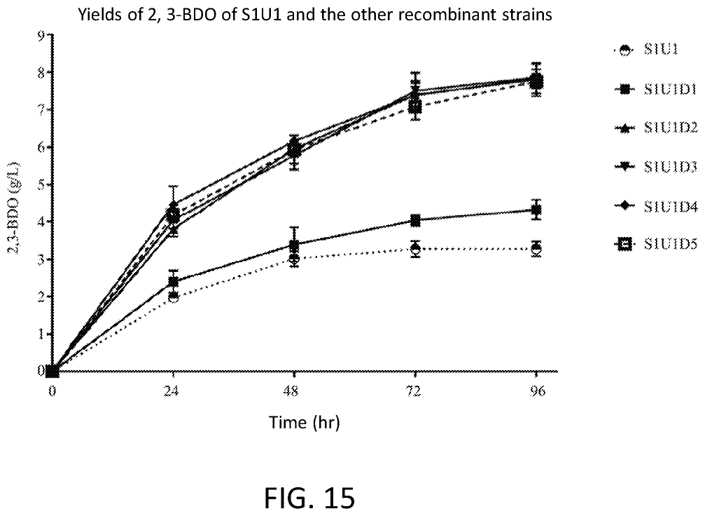

The recombinant strains of each gene are cultivated in M9 culture medium consisting of 5% glucose in a temperature of 30° C. and are sampled in 24^{th}, 48^{th}, 72^{nd}, and 96^{th} hours, and yields of 2,3-BDO produced from the recombinant strains of different genes by the leaven are analyzed by using TLC-vanillin method. Referring to FIGS. 14A and 14B, an analysis result of FIGS. 14A and 14B is quantized by the Image J software, listed in Table 6, and represented by the curve line, as shown in FIG. 15.

Measured yield of 2,3-BDO from S1U1D5 at different times

| | Yield of 2,3-BDO (g/L) | | | | | |
|---|---|---|---|---|---|---|
| Time (hours) | S1U1 | S1U1D1 | S1U1D2 | S1U1D3 | S1U1D4 | S1U1D5 |
| 0 | 0 ± 0 | 0 ± 0 | 0 ± 0 | 0 ± 0 | 0 ± 0 | 0 ± 0 |
| 24 | 1.97 ± 0.11 | 2.4 ± 0.29 | 3.8 ± 0.2 | 4.04 ± 0.23 | 4.46 ± 0.49 | 4.18 ± 0.17 |
| 48 | 3.01 ± 0.2 | 3.39 ± 0.45 | 5.96 ± 0.21 | 5.77 ± 0.38 | 6.16 ± 0.08 | 5.94 ± 0.38 |
| 72 | 3.27 ± 0.21 | 4.03 ± 0.15 | 7.4 ± 0.32 | 7.5 ± 0.49 | 7.39 ± 0.38 | 7.08 ± 0.35 |
| 96 | 3.27 ± 0.2 | 4.32 ± 0.27 | 7.81 ± 0.44 | 7.86 ± 0.22 | 7.83 ± 0.39 | 7.75 ± 0.17 |

With reference to FIG. 15 and Table 6, the yield of 2,3-BDO produced from S1U1 after 60^{th} hours decreases slowly, and the yield of 2,3-BDO produced from S1U1D1 after 90^{th} hours increases slowly. Compared with S1U1, the yields of 2,3-BDO produced from S1U1D2, S1U1D3, S1U1D4, and S1U1D5 increase greatly, for example, the yields of 2,3-BDO produced from S1U1D2, S1U1D3, S1U1D4, and S1U1D5 increase greatly from the beginning to 96^{th} hours, wherein the yields of 2,3-BDO produced from S1U1D2, S1U1D3, S1U1D4, and S1U1D5 after cultivating the recombinant strains for 48 hours are 5.77 g/L to 6.16 g/L which are 91.7% to 105% more than 3.01 g/L of the yield of S1U1. After cultivating the recombinant strains for 72 and 96 hours, the yields of 2,3-BDO are 7.08 g/L to 7.86 g/L which are 117% to 140% more than 3.27 g/L of the yield of S1U1.

In a fifth embodiment, values of power of hydrogen (pH) of fermentations of the recombinant strains are compared.

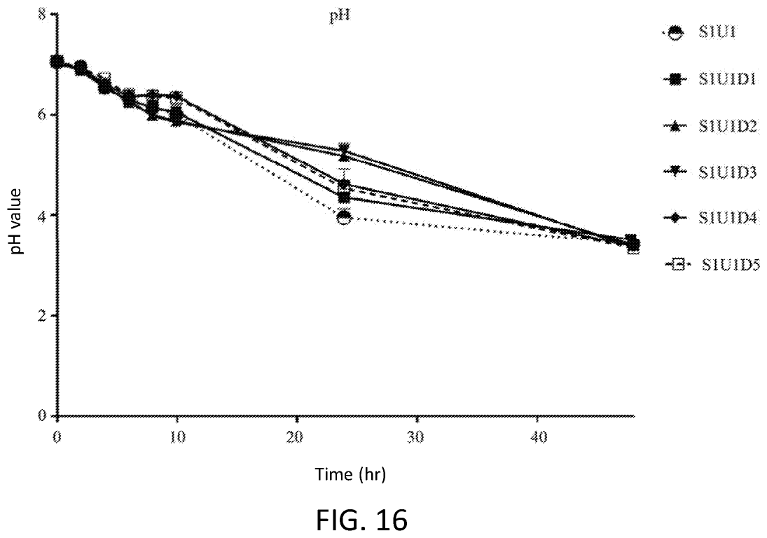

The recombinant strains of each gene are cultivated in M9 culture medium consisting of 5% glucose in a temperature of 30° C. and are sample to measure the values of pH of the bacterial solutions which are shown in FIG. 16, wherein after cultivating the recombinant strains in the first six hours, the values of pH of the recombinant strains are similar, but after cultivating the recombinant strains in 24^{th} hours, the values of pH of the recombinant strains are quite different. For example, the values of pH of S1U1D3 and S1U1D2 are highest (such as more than pH 5), and a value of pH of S1U1 is lowest (such as pH 4), so the recombinant strains slow down acidification of the leaven.

Referring to FIG. 16, after cultivating the recombinant strains in 48th hours, the values of pH of the recombinant strains decrease to 3 to 4. As shown in FIGS. 15-16 and Table 6, the yields of 2,3-BDO produced from S1U1D2, S1U1D3, S1U1D4, and S1U1D5 in a low pH fermentation are high and are not influenced by the low pH fermentation. For instance, after cultivating the recombinant strains S1U1D2, S1U1D3, S1U1D4, and S1U1D5 for 48 hours, the values of pH of the recombinant strains decrease below 4, but the yields of 2,3-BDO produced from S1U1D2, S1U1D3, S1U1D4, and S1U1D5 are higher than the yield of 2,3-BDO produced from S1U1, wherein the yields of 2,3-BDO produced from S1U1D2, S1U1D3, S1U1D4, and S1U1D5 are 5.77 g/L to 6.16 g/L. After cultivating the recombinant strains S1U1D2, S1U1D3, S1U1D4, and S1U1D5 for 72 hours and 96 hours, the yields of 2,3 produced from S1U1D2, S1U1D3, S1U1D4, and S1U1D5 are 7.08 g/L to 7.86 g/L which are twice more than the yield of 2,3-BDO produced from S1U1.

The above description is made on embodiments of the present invention. However, the embodiments are not intended to limit scope of the present invention, and all equivalent implementations or alterations within the spirit of the present invention still fall within the scope of the present invention.

What is claimed is:

1. A recombinant microorganism for producing 2,3-butanediol consisting of selecting at least three groups from uridine diphosphate glucose phosphate uroglycan transferase gene (galU), acetyl alcohol dehydrogenase gene (acoA), acetyl phosphate transferase gene (pta), adenosine glucosylphosphate transferase gene (glgC), lactose dehydrogenase gene (ldhA), and phosphodiesterase gene (pdeC) which had gene modification.

2. The recombinant microorganism as claimed in claim 1 further consisting of the galU, the acoA, and the pta which had gene modification.

3. The recombinant microorganism as claimed in claim 1 further consisting of the galU, the acoA, the pta, and the glgC which were modified.

4. The recombinant microorganism as claimed in claim 1 further consisting of the galU, the acoA, the pta, the glgC, and the IdhA which had gene modification.

5. The recombinant microorganism as claimed in claim 1 further consisting of the galU, the acoA, the pta, the glgC, the ldhA, and the pdeC which had gene modification.

6. The recombinant microorganism as claimed in claim 1, wherein the genetic modification is any one of gene suppression, gene deletion and gene silencing.

7. The recombinant microorganism as claimed in claim 6, wherein the genetic modification is gene deletion.

8. The recombinant microorganism as claimed in claim 1, wherein the recombinant microorganism is Klebsiella.

9. The recombinant microorganism as claimed in claim 1, wherein a yield of 2,3-butanediol of the recombinant microorganism is higher than a yield of 2,3-butanediol of a wild-type microorganism.

10. The recombinant microorganism as claimed in claim 1, wherein recombinant microorganism is safer than a wild-type microorganism.

11. The recombinant microorganism as claimed in claim 1, wherein a growing rate of the recombinant microorganism is equal to a growing rate of a recombinant microorganism which was not modified.

12. The recombinant microorganism as claimed in claim 1, wherein the recombinant microorganism produces the 2,3-butanediol in an acidic environment.

13. A method for production of the 2,3-butanediol comprising cultivating the recombinant microorganism of claim 1 and separating the 2, 3-butanediol from the culture medium of claim 1.
